(19) United States
(12) Patent Application Publication
(10) Pub. No.: US 2013/0190632 A1
(43) Pub. Date: Jul. 25, 2013

(54) AUTOREGULATION MONITORING

(71) Applicants: Robert A. Baruch, Ellicott City, MD (US); Ken M. Brady, Sugar Land, TX (US); Ronald B. Easley, League City, TX (US); Craig G. Rusin, Houston, TX (US)

(72) Inventors: Robert A. Baruch, Ellicott City, MD (US); Ken M. Brady, Sugar Land, TX (US); Ronald B. Easley, League City, TX (US); Craig G. Rusin, Houston, TX (US)

(21) Appl. No.: 13/748,833

(22) Filed: Jan. 24, 2013

Related U.S. Application Data
(60) Provisional application No. 61/590,378, filed on Jan. 25, 2012.

(51) Int. Cl. A61B 5/00; A61B 5/03; A61M 16/12; A61B 5/0205; A61M 16/00; A61M 16/20

(52) U.S. Cl. CPC A61B 5/4064; A61M 16/0057; A61M 16/20; A61M 16/12; A61B 5/0205; A61B 5/031; A61B 5/7275
USPC 600/484

(57) ABSTRACT

A method may include controlling a ventilator to introduce mean airway pressure (MAP) variations in a patient to induce slow waves of substantially fixed amplitude and period to the patient. The method may also include analyzing arterial blood pressure in the patient with respect to the MAP variations and determining, based on the analyzing, whether an autoregulatory mechanism associated with the patient's brain is operating properly.

### RELATED APPLICATION

This application claims priority under 35 U.S.C. §119 based on U.S. Provisional Patent Application No. 61/590,378, filed Jan. 25, 2012, the disclosure of which is hereby incorporated herein by reference.

### BACKGROUND INFORMATION

Autoregulation refers to the maintenance of constant cerebral blood flow across a range of cerebral perfusion pressures. Autoregulation is a homeostatic mechanism that protects the brain from excessive or inadequate blood flow. Monitoring autoregulation may be useful in several clinical scenarios where perfusion of the brain may be compromised, such as after trauma to the head, during cardiopulmonary bypass, in the setting of sepsis, during shock from premature birth, etc. Patients with impaired autoregulation are more likely to die, and more likely to suffer permanent neurologic disability. Autoregulation monitoring can be used to delineate care practices that enhance the ability of the brain to regulate its own blood flow. However, conventional autoregulation monitoring often takes a considerable amount of time.

### BRIEF DESCRIPTION OF THE DRAWINGS

FIG. 1A provides graphs illustrating experimental monitoring data associated with a piglet;

FIG. 1B is a graph illustrating the generation of a pressure reactivity index based on the experimental data in FIG. 1A via a linear correlation between the arterial blood pressure and intracranial pressure;

FIG. 1C provides graphs illustrating additional experimental monitoring data associated with the piglet;

FIG. 1D is a graph illustrating the pressure reactivity index based on the data in FIG. 1C;

FIG. 2 illustrates exemplary components of an intracranial pressure waveform;

FIG. 3 illustrates experimental data for a piglet on bypass;

FIG. 4 illustrates an exemplary environment in which systems and methods described herein may be implemented;

FIG. 5 illustrates an exemplary configuration of components implemented in the ventilator of FIG. 4;

FIG. 6 illustrates an exemplary configuration of components implemented in the monitoring device of FIG. 4;

FIG. 7 is a flow diagram illustrating exemplary processing by various devices illustrated in FIG. 4;

FIG. 8 illustrates various waveforms associated with monitoring autoregulation for animal subjects in an experimental study;

FIG. 9 illustrates measurements made in the experimental study to define the lower limits of autoregulation;

FIGS. 10A-10C illustrate different metrics against the lower limit of autoregulation;

FIG. 11 illustrates the precision associated with various metrics of autoregulation;

FIG. 12 illustrates normalizing various metrics associated with the lower limit of autoregulation; and

FIG. 13 illustrates the accuracy associated with various metrics of autoregulation.

### DETAILED DESCRIPTION OF PREFERRED EMBODIMENTS

The following detailed description refers to the accompanying drawings. The same reference numbers in different drawings may identify the same or similar elements. Also, the following detailed description does not limit the invention. Instead, the scope of the invention is defined by the appended claims and their equivalents.

Implementations described herein provide methods, systems and computer program products for monitoring cerebrovascular autoregulation to optimize hemodynamic management for patients. In one implementation, repetitive, hemodynamic oscillations (referred to as “slow waves”) are induced using a ventilator. For example, slow waves may be induced in a patient using the ventilator to vary the mean airway pressure. These induced “slow waves” allow for precise measurements with respect to autoregulation in a very short period of time. The measurements may also allow medical personnel to quickly ascertain certain conditions and optimize care for a patient.

Autoregulation monitoring examines the reaction, (or lack thereof) of the brain vasculature to a change in arterial blood pressure. When blood pressure changes, the blood flow increase should be opposed by the autoregulatory mechanism. This is done by vascular constriction, which decreases blood volume in the cranial vault. Therefore, autoregulation can be monitored by examining the relationship between arterial blood pressure and cerebral blood flow, or arterial blood pressure and cerebral blood volume. Several different surrogates of both cerebral blood flow and cerebral blood volume have been used for autoregulation monitoring. Some examples are shown in table 1 below.

TABLE 1

| Monitor Name | Modality Used | Additional Information |
|---|---|---|
| Mean Velocity Index (Mx) | Ultrasound: flow velocity is | The first monitor of |
| | used as a surrogate for | autoregulation. Has been |
| | cerebral blood flow | used in head trauma and |
| | | bypass. |
| Pressure Reactivity Index | Intracranial pressure is used | Robust data showing link |
| (PRx) | as a surrogate for cerebral | between PRx and outcome. |
| | blood volume | |
| Laser-Doppler Index (LDx) | Laser-Doppler: cortical red | Limited clinical use because |
| | cell flux is used as a | of invasive nature of Laser- |
| | surrogate for cerebral blood | Doppler. |
| | flow | |
| Cerebral Oximetry Index | Near-Infrared Spectroscopy: | Has been studied in |
| (COx) | cortical oximetry is used as | cardiopulmonary bypass. |
| | a surrogate for cerebral | |
| | blood flow | |
| Hemoglobin Volume Index | Near-Infrared Spectroscopy: | In theory, this index is less |
| (HVx) | optical density of 810 nm | confounded than the COx |
| | reflectance Spectroscopy is | by various changes in |
| | used as a surrogate for | patient physiology. |
| | cerebral blood volume | |
| Vittamed | Uses time of flight | This analysis is done in the |
| | ultrasound as a surrogate for | frequency domain, by |
| | cerebral blood volume | examining phase angles |
| | | between arterial blood |
| | | pressure and cerebral blood |
| | | volume waves. |

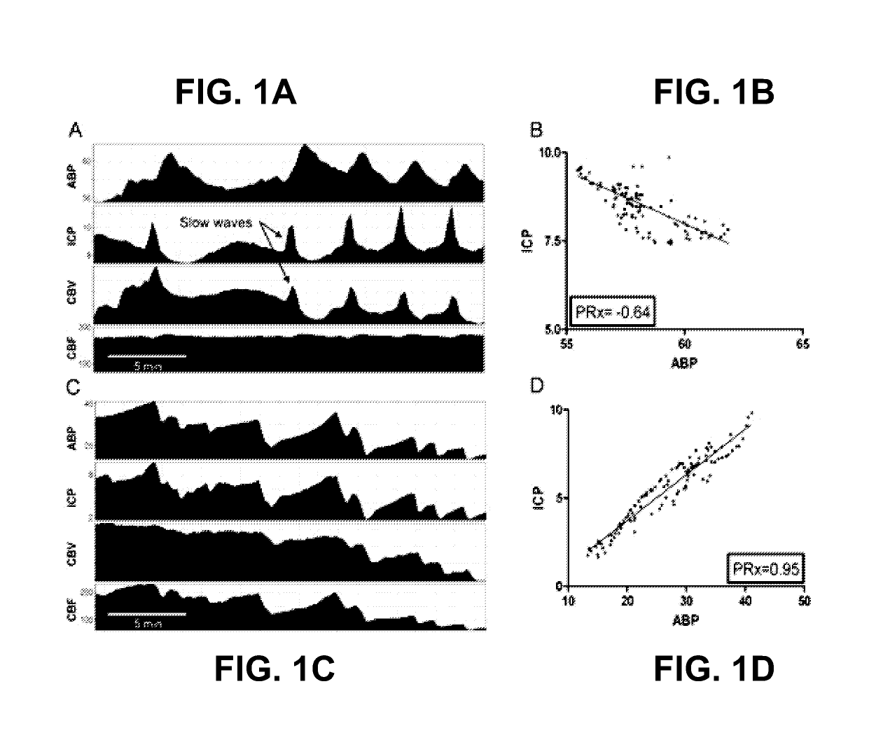

Regardless of the modality used to measure autoregulation, a change in arterial blood pressure is needed to examine the autoregulatory reaction. When autoregulation is intact, changes in pressure cause vascular reactivity, as shown in FIG. 1A. FIG. 1B illustrates an accompanying example of a pressure reactivity (PRx) calculation by simple correlation of arterial blood pressure and intracranial pressure from the data of FIG. 1A.

When autoregulation is intact, cerebral blood volume changes in opposition to changes in arterial blood pressure. Therefore, autoregulation is considered reactive, and gives a negative correlation. In the frequency domain, such a negative correlation would result in a large phase angle difference between the two waves.

FIGS. 1C and 1D show the result of failed autoregulation, such as when the cerebral vasculature is passive to changes in arterial blood pressure. In the passive state, cerebral blood volume and flow changes are in phase with arterial blood pressure changes, which yields a positive linear correlation between them. Therefore, without a change in arterial blood pressure, it is difficult or impossible to make a meaningful assessment of autoregulation. There are multiple wave components, operating at different frequencies, which summate to yield the arterial blood pressure waveform. Of all these frequencies, it has been found that the “slow wave” frequencies are best used for monitoring autoregulation.

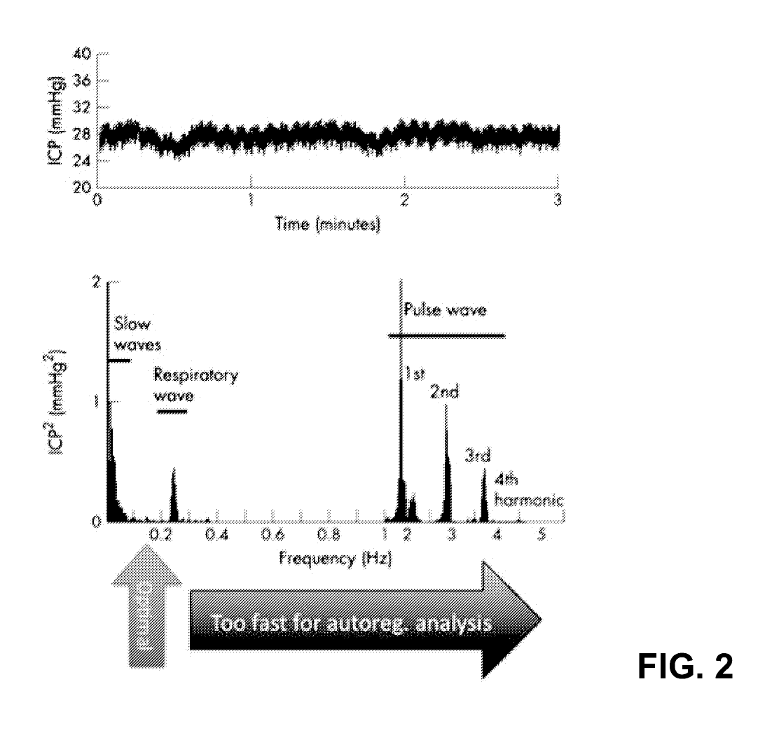

FIG. 2 illustrates a Fourier transform of the intracranial pressure (ICP) waveform. Referring to FIG. 2, three prominent wave components are shown: 1) the pulse frequency, 2) the respiratory frequency, and 3) the so-called slow wave frequency. Pulse and respiratory rhythms are, by comparison to slow wave rhythms, much more regular in both periodicity and amplitude. The etiology of slow wave activity is not well understood.

It is generally known that pulse and respiratory waves are too fast for the autoregulatory mechanism. One current technology discussed above in Table 1 used by Vittamed Technologies uses respiratory waves for this purpose, but requires mechanical ventilation with a fixed low rate appropriate only in adult patients. Most metrics of autoregulation use the slow wave frequency because vascular responses are full in the slow bandwidth, effectively acting as a high-pass filter for cerebral blood flow constraint. Because slow waves are not fixed in period or amplitude, many measurements of autoregulation must be taken and averaged together to cancel noise introduced by variability.

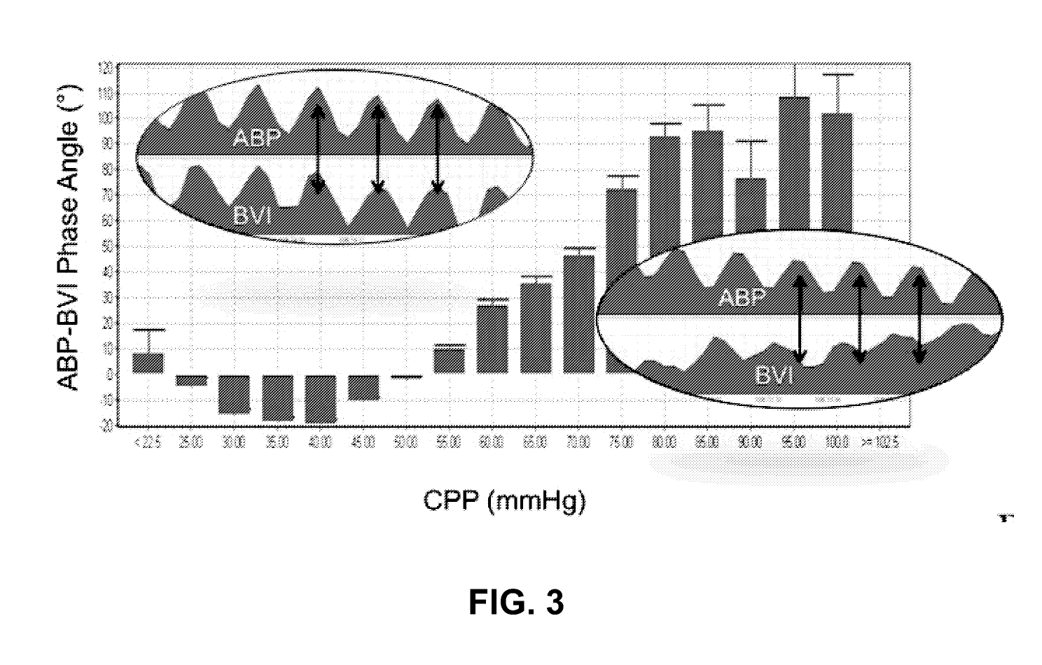

In another technology/methodology, slow waves are generated for monitoring during cardiopulmonary bypass. By oscillating the flow pattern of the bypass pump, the arterial blood pressure is manipulated to have the same input wave. This technology has been tested by comparing the phase angle between arterial blood pressure (ABP) and cerebral blood volume (e.g., a blood volume index (BVI)) at the input wave frequency, as illustrated in FIG. 3.

In accordance with an exemplary implementation described below, an ideal slow wave for measuring autoregulation is generated. The slow wave is regular in period, fixed in amplitude, and slightly slower in frequency than the normal adult respiratory rate (as indicated by the arrow labeled “optimal” in FIG. 2). That is, implementations described below generate a slow wave that is relatively fast to allow frequent measurements, but still slow enough for a complete autoregulatory response.

Some advantages of a manufactured wave are that the frequency can be chosen to yield the most rapid and precise measurements of autoregulation. Such a bypass model gives useful autoregulation information within, for example, five minutes, as compared to a minimum of 30 minutes for the spontaneous wave analysis method. Additional advantages are that the measurements are more precise because analysis only takes place at the input frequency. Other physiologic events that can impact on cerebral blood flow or volume do not occur in repetitive cycles in this frequency. Noise, which is also a recurring problem with the spontaneous slow wave method, is virtually eliminated by using a fixed input wave.

As described above, in some technologies, a bypass pump has been used to manufacture slow waves to measure autoregulation. A drawback with this methodology is the need for the patient to be on bypass. Many patient populations not on bypass would also benefit from autoregulation monitoring. These populations include, but are not limited to: the pre-term neonate, patients with septic shock, and neurosurgical patients, especially patients with traumatic brain injury. Therefore, it has been found that it would be beneficial to have a safe way to induce repetitive slow wave activity in these patients to increase the precision of autoregulation monitoring, as well as decrease the time needed for useful autoregulation monitoring.

In accordance with exemplary implementations, changes in mean airway pressure have been found to cause changes in arterial blood pressure by impeding and facilitating the return of blood to the heart. This is the cause of respiratory variation seen in the arterial blood pressure of patients on mechanical ventilation. As described above, one technology uses the respiratory frequency wave to measure autoregulation in the brain and does not require a continuous arterial blood pressure input. One downside to this method is the need for a very slow ventilation rate, which may not be possible for all patients, especially infants.

In accordance with embodiments described herein, ventilator functions associated with normal ventilation are separated from functions associated with generating slow waves. For example, the mechanical ventilation function of the ventilator is separated from the function associated with the induction of slow wave activity by creating separate wave components, at separate frequencies specific for their desired functions. To explain examples of this process, some basic ventilator terminology is defined in Table 2 below.

TABLE 2

| Term | Definition | Considerations |
|---|---|---|
| Rate (r) | The breathing rate | Normal infant rate is around 25/min, Normal adult rate |
| | (breaths/min) | is around 8/min. |
| Tidal Volume | The volume of gas | Normal V_{T} ranges 6-10 cc/kg. |
| (V_{T}) | moved with each | |
| | breath (liters) | |
| Minute | Rate X Tidal | MV describes the flow of air through the lungs. This is |
| Ventilation | Volume (liters/min) | the main determinant of CO₂ removal, but does not |
| (MV) | | determine oxygenation. |
| Peak | The maximum | High PIP indicates poor lung compliance caused by |
| Inspiratory | pressure achieved | tissue water, inflammation, lack of biological |
| Pressure (PIP) | during a | surfactants, etc. High PIP is injurious. |
| | mechanically-driven | |
| | respiratory cycle. | |
| | (cm H₂0) | |
| Positive End | The pressure of the | Optimization of PEEP is central to achieving adequate |
| Expiratory | airway circuit at the | but not excessive inflation of a diseased lung |
| Pressure | end of exhalation, | (“recruitment”). Normal PEEP is 5-8 cm H₂O |
| (PEEP) | just prior to a | |
| | mechanical breath | |
| | delivery | |
| Mean Airway | The time integration | This is affected by PIP, PEEP, and the relative |
| Pressure | of airway pressure. | inspiratory and expiratory durations. Normally |
| (MAP) | | expiration is twice the duration of inspiration, so |
| | | PEEP changes affect MAP more than PIP changes. |
| | | MAP is the main determinant of lung “recruitment” |
| | | which allows gas-capillary interactions and |
| | | oxygenation. |

In one exemplary embodiment, mean airway pressure (MAP) oscillations at low frequency are generated with normal minute ventilation. For example, consider a patient on mechanical ventilation at normal settings for a 20 kilogram (kg) child: Rate 18 breaths/minute (min), Tidal Volume 160 cubic centimeters (cc), Minute Ventilation 2.8 liters (L)/min. In an exemplary scenario, assume that PEEP is set to 6 centimeters (cm) H₂O, and because of a moderately diseased lung, the PIP is 25 cm H₂O. The MAP, however, may be only 11 cm H₂O, because the majority of time is spent in exhalation. The respiratory wave in this child's arterial blood pressure tracing is at a frequency of 0.3 Hertz (Hz) (i.e., 18 breaths/min divided by 60 seconds/min), which is faster than the filtering effect of autoregulation. Therefore, there is minimal phase shift between blood volume changes in the brain and the ventilator cycle when measured at the respiratory cycle. As a result, the respiratory rate is not useful to measure autoregulation, but is required to ventilate the child.

In accordance with an exemplary implementation, the ventilator is used to induce a second wave in a patient at a frequency other than the respiratory rate. In such an implementation, the second wave does not impact the ventilator functions and does not affect the physiology of the patient with respect to the ventilator function. That is, the patient's ventilation stays constant and a second wave is generated at a modulating frequency that allows for precise autoregulation measurements to be made.

For example, in accordance with one implementation, the minute ventilation settings of the ventilator are left untouched, but a variation in the PEEP is induced in a repetitive cycle at a lower frequency than the respiratory frequency. For example, the variation in PEEP may be safely done at an amplitude of 1-2 cm H₂O over a period of 30 seconds (i.e., a frequency of approximately 0.03 Hz), which would be well within safe PEEP settings. The resultant change in mean airway pressure causes a second slow oscillation in arterial blood pressure—the first being caused by the minute ventilation at 0.3 Hz and the second being caused by the PEEP oscillation at 0.03 Hz. In this implementation, the analysis of autoregulation that follows is performed only at the 0.03 Hz frequency, and is unaffected by the minute ventilation. In addition, the minute ventilation is unaffected by the PEEP oscillation. That is, the ventilator is able to perform its ventilation function and the patient suffers no adverse effects.

Because PEEP is a major determinant of intrathoracic pressure, small changes in PEEP are sufficient to cause changes in arterial blood pressure. However, the relationship is not linear, and is dependent on several patient and situational factors.

In another exemplary embodiment, low ventilator rates may be used when minute ventilation is not needed. For example, patients are often supported with devices to remove CO₂ and rest the lung. For instance, the Novalung® has become increasingly popular for this purpose. Prior to this treatment, full bypass support was used for this purpose. Regardless of the modality of support used, when CO₂ is removed from the blood extra-corporeally, there is no need for minute ventilation. In this instance, the lung is often “rested” at low rates, low tidal volumes and high PEEP. In accordance with one implementation, the ventilator may be optimized for the creation of slow waves and these critically-ill patients with total respiratory failure could benefit from autoregulation monitoring. As an example, one form of optimization would be to provide a slow ventilator rate of 1-2 breaths/min, between “rest” PIP pressure of 20 and PEEP of 10.

It should be understood that the two implementations/examples described above are not inclusive of all the ways that a ventilator can be used to generate a slow wave at a frequency suitable for autoregulation monitoring. In addition, while only conventional ventilation has been discussed, embodiments described herein can be applied to High Frequency Oscillation-type ventilation, Airway-Pressure Release ventilation, and other non-conventional ventilation modes. In each case, a low frequency oscillation of mean airway pressure is generated that creates slow waves in the arterial blood pressure, but does not impact minute ventilation.

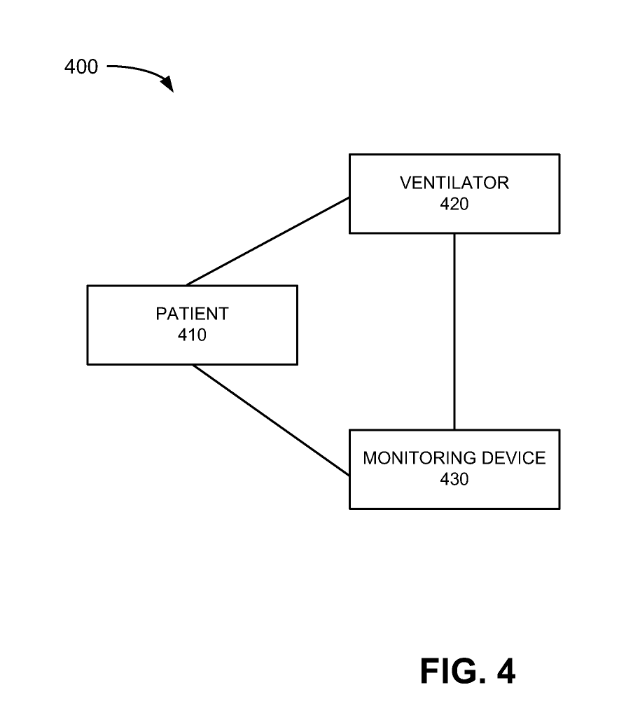

As described above, a ventilator may be used to induce slow waves in the patient. For example, FIG. 4 is a block diagram of an exemplary environment in which systems and methods described herein may be implemented. Referring to FIG. 4, environment 400 may include a patient 410, a ventilator 420 and a monitoring device 430.

Patient 410 may represent any person (i.e., an adult or child) that may be in a state of medical distress or has sustained an injury. Ventilator 420 may be a ventilator used to provide ventilation to patient 410. Ventilator 420 may include conventional controls used to control, for example, respiratory rate, tidal volume, minute ventilation, PIP, PEEP and MAP. As described above, in an exemplary implementation, ventilator 420 may be used to provide mechanical ventilation functions for patient 410, while simultaneously creating slow waves in patient 410.

Monitoring device 430 may include a device used to continuously monitor various parameters associated with patient 410. In an exemplary implementation, monitoring device 430 may receive data from patient 410 and/or equipment connected to patient 410 to determine whether patient 410's brain is properly autoregulating (e.g., within normal ranges). This information may then be used to control and/or regulate various parameters, such as ABP, to provide the proper blood flow to patient 410 to allow patient 410's brain to autoregulate properly.

Exemplary environment 400 illustrated in FIG. 4 is provided for simplicity. It should be understood that a typical environment may include more or fewer devices than illustrated in FIG. 4. For example, in some instances, a ventilator controller may be a separate element from ventilator 420. In still other implementations, monitoring device 430 may be used to set/control ventilator 420. In addition, in some implementations, the functions described below as being performed by multiple devices in environment 400 may be performed by a single device. For example, in some implementations, the functions performed by ventilator 420 and monitoring device 430 may be combined into a single device. In addition, in an alternative implementation, some elements may not be used.

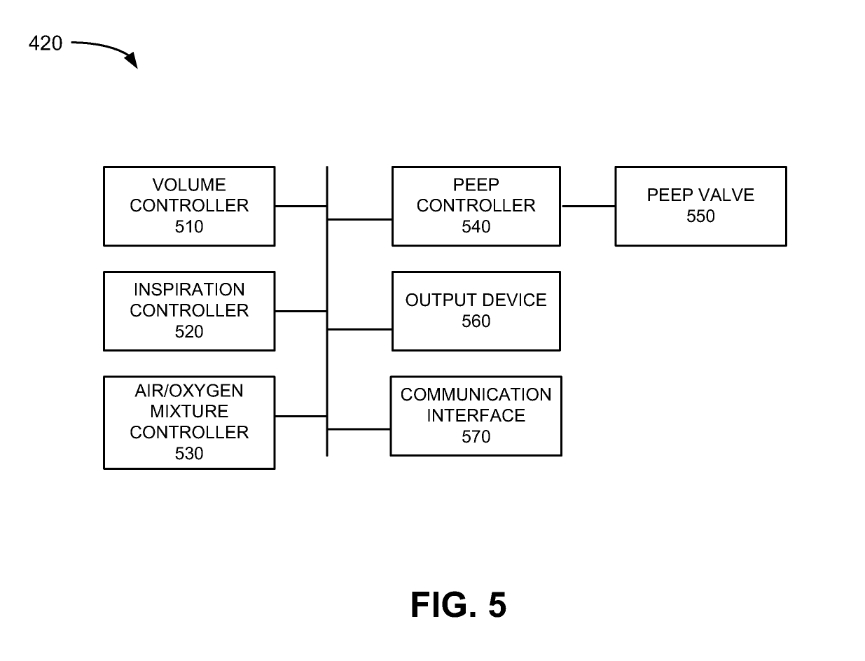

FIG. 5 illustrates an exemplary configuration of components included in ventilator 420. Referring to FIG. 5, ventilator 420 may include volume controller 510, inspiration controller 520, air/oxygen mixture controller 530, PEEP controller 540, PEEP valve 550, output device 560 and communication interface 570. The components illustrated in FIG. 5 are exemplary only. It should be understood that ventilator 420 may include more or fewer components than illustrated in FIG. 5. In addition, in some implementations, the functions described below as being performed by multiple components in ventilator 420 may be performed by a single component.

Volume controller 510 may control the volume of air/oxygen provided to patient 410. For example, volume controller 510 may interface with one or more pumps and valves (not shown) to provide the designated volume of air/oxygen to patient 410.

Inspiration controller 520 may control the airway pressure for patient 410. For example, inspiration controller 520 may control an adjustable valve to provide the desired inspiration to patient 410. Air/oxygen mixture controller 530 may control the mixture of air and oxygen provided to patient 410. For example, air/oxygen mixture controller 520 may interface with valves (not shown) to control the air-oxygen mixture.

PEEP controller 540 may control PEEP provided to patient 410. For example, PEEP controller 540 may interface with PEEP valve 550 to provide the desired PEEP. In an exemplary implementation, PEEP controller 540 may be programmable to modulate the PEEP provided to patient 410 to generate a slow wave. For example, PEEP controller 540 may control PEEP valve to oscillate the PEEP between an upper and lower value corresponding to a sine wave pattern, as described in detail below.

Output device 560 may include a mechanism that outputs information to medical personnel, including a display, a printer, a speaker, etc. For example, output device 560 may include a display screen (e.g., a liquid crystal diode (LCD) display or another type of display) that provides information to a medical personnel regarding patient 410.

Communication interface 570 may include any transceiver that enables ventilator 420 to communicate with other devices and/or systems. For example, communication interface 570 may communicate with other devices coupled to patient 410, such as monitoring device 430. Communication interface 570 may also include a modem or an Ethernet interface to a LAN. Alternatively, communication interface 570 may include other mechanisms for communicating via a network (not shown).

In some implementations, all or some of the control devices illustrated in FIG. 5, such as volume controller 510, inspiration controller 520, air/oxygen mixture controller 530 and PEEP controller 540 may be implemented as electromechanical devices. In other implementations, all or some of the control devices illustrated in FIG. 5 may be implemented via computer hardware and/or software. For example, each of the components illustrated in FIG. 5 may include one or more processors, microprocessors, application specific integrated circuits (ASICs), field programmable gate arrays (FPGAs), or other processing logic that controls various functions of ventilator 420, such as PEEP, via software instructions. In this case, the software instructions may control the processor/processing logic to provide the desired functions, such as oscillate the PEEP to generate slow waves, as described above. Alternatively, hard-wired circuitry may be used in place of or in combination with software instructions to implement processes described herein. Thus, implementations described herein are not limited to any specific combination of electromechanical devices, hardware circuitry and software.

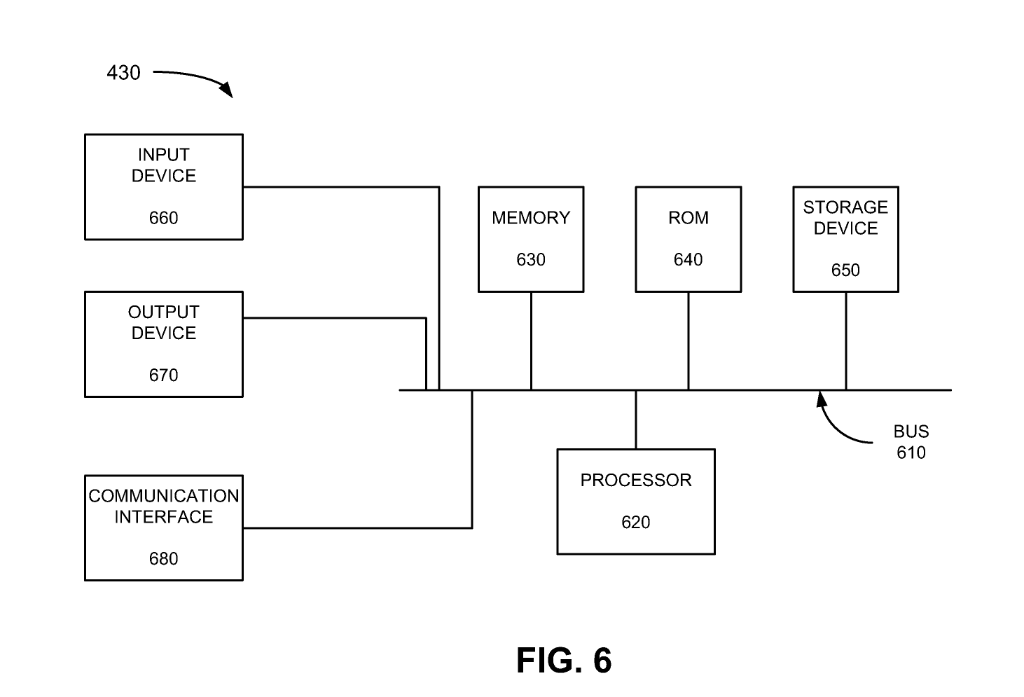

FIG. 6 illustrates an exemplary configuration of monitoring device 430. In some implementations, ventilator 420 may include similar components and/or be configured in a similar manner. Referring to FIG. 6, monitoring device 430 may include bus 610, processor 620, main memory 630, read only memory (ROM) 640, storage device 650, input device 660, output device 670, and communication interface 680. Bus 610 may include a path that permits communication among the elements of monitoring device 430.

Processor 620 may include a processor, microprocessor, application specific integrated circuit (ASIC), field programmable gate array (FPGA) or processing logic that may interpret and execute instructions. Memory 630 may include a random access memory (RAM) or another type of dynamic storage device that may store information and instructions for execution by processor 620. ROM 640 may include a ROM device or another type of static storage device that may store static information and instructions for use by processor 620. Storage device 650 may include a magnetic and/or optical recording medium and its corresponding drive.

Input device 660 may include a mechanism that permits an operator to input information to monitoring device 430, such as a keyboard, control keys, a mouse, a pen, voice recognition and/or biometric mechanisms, etc. Input device 660 may also include one or more control buttons, knobs or keypads to allow an operator to set various parameters with respect to controlling environment 400.

Output device 670 may include a mechanism that outputs information to the operator, including a display, a printer, a speaker, etc. For example, output device 670 may include a display screen (e.g., a liquid crystal diode (LCD) display or another type of display) that provides information to medical personnel regarding patient 410.

Communication interface 680 may include any transceiver that enables monitoring device 430 to communicate with other devices and/or systems. For example, communication interface 680 may communicate with other devices coupled to patient 410, such as ventilator 420. Communication interface 680 may also include a modem or an Ethernet interface to a LAN. Alternatively, communication interface 680 may include other mechanisms for communicating via a network (not shown).

Monitoring device 430 may perform processing associated with monitoring slow wave induced into patient 410, as described above. According to an exemplary implementation, monitoring device 430 may perform these operations in response to processor 620 executing sequences of instructions contained in a computer-readable medium, such as memory 630. A computer-readable medium may be defined as a physical or logical memory device.

The software instructions may be read into memory 630 from another computer-readable medium, such as data storage device 650, or from another device via communication interface 680. The software instructions contained in memory 630 may cause processor 620 to perform processes that will be described later. Alternatively, hard-wired circuitry may be used in place of or in combination with software instructions to implement processes described herein. Thus, implementations described herein are not limited to any specific combination of hardware circuitry and software.

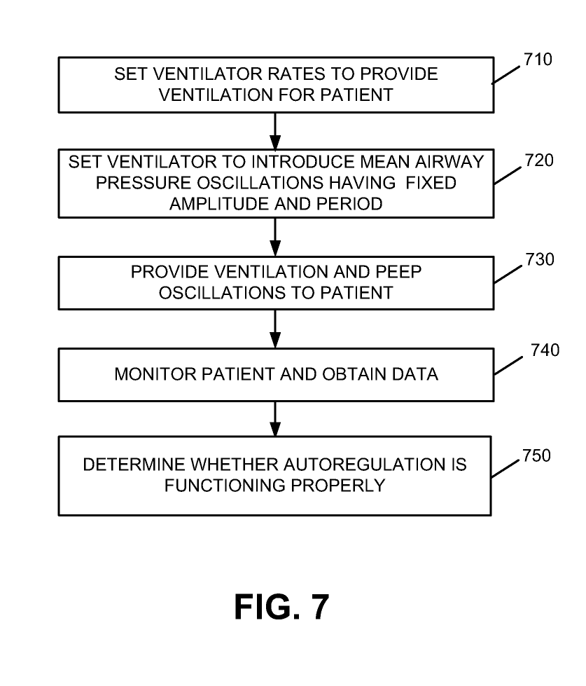

FIG. 7 is a flow diagram illustrating exemplary processing associated with generating or inducing slow waves to patient 410 via ventilator 420. In this example, assume that patient 410 is on ventilator 420 and requires minute ventilation. Processing may begin with a health care provider setting ventilator 420 to provide ventilation for patient 410 (block 710). For example, continuing with the example described above in which patient 410 is a child weighing 20 kg, ventilator 420 may be set to provide 18 breaths/min, Tidal Volume of 160 cc, Minute Ventilation of 2.8 L/min, PEEP of 6 cm H₂O, PIP of 25 cm H₂O, and MAP of 11 cm H₂O. For example, volume controller 510, inspiration controller 520 and/or other controllers associated with ventilator 420 may be used to provide these parameters associated with the ventilation function of ventilator 420. These ventilation settings may be needed to ventilate patient 410 in accordance with medical personnel's evaluation of patient 410, but may not be useful for measuring autoregulation of patient 410.

In accordance with an exemplary implementation, ventilator 420 may also be set to introduce flow variations, such as MAP oscillations, that have a fixed amplitude and period to create a slow wave in patient 410's brain (block 720). For example, medical personnel may set PEEP controller 540 to oscillate PEEP in patient 410 at an amplitude of 1-2 cm H₂O over a period of 30 seconds (i.e., a frequency of about 0.03 Hz). In one implementation, the PEEP controller 540 may be programmed to oscillate the PEEP between the lower and higher PEEP values in a sine wave pattern. In other implementations, other oscillating patterns may be used. In each case, ventilator 420 may then provide its mechanical ventilation functions associated with patient 410, while simultaneously creating a slow wave useful for autoregulation monitoring (block 730).

Monitoring device 430 may then monitor various parameters and/or obtain data associated with patient 410 at the input wave frequency to determine whether patient 410's brain is responding to the fixed oscillations (block 740).

For example, monitoring device 430 may monitor the ABP and cerebral blood volume in patient 410's brain at the frequency of the induced slow waves, e.g., approximately 0.03 Hz in this example, to determine the autoregulation state of patient 410's brain. For example, the blood volume in the brain of patient 410 may be negative phase shifted (i.e., the peak occurs earlier) with respect to the blood pressure (e.g., ABP) by some amount (e.g., 10 degrees to more than 150 degrees) when the brain's autoregulatory mechanism is intact. In an exemplary implementation, monitoring device 430 may use intracranial pressure (ICP) as a surrogate for cerebral blood volume. In this implementation, monitoring device 430 may monitor ICP in the frequency domain at the frequency of the induced slow waves, while also monitoring ABP at the frequency of the induced slow waves. Monitoring device 430 may also continuously output waveforms via output device 670 illustrating ABP and ICP of patient 410 at the frequency of the induced slow waves.

The information gathered by monitoring device 430 may then be analyzed to identify whether patient 410's autoregulatory mechanism is functioning properly (block 750). For example, medical personnel may view the ABP and ICP waveforms to determine if the blood volume (or ICP) in patient 410's brain is 0° phase shifted from the input blood volume wave.

If the ICP and ABP waveforms are 0° phase shifted with respect to each other (i.e., are essentially in phase), autoregulation of patient 410's brain may not be operating. Monitoring device 430 and/or personnel associated with monitoring patient 410 may then set various parameters and/or administer various drugs to patient so that autoregulation will function properly. If, however, the ICP waveform is negative phase shifted (i.e., the peak occurs earlier) with respect to the ABP waveform by some amount (e.g., 10 degrees to more than 150 degrees) then the brain's autoregulatory mechanism may be considered to be intact or functioning properly.

In some implementations, monitoring device 430 may automatically analyze the ICP and ABP waveforms and output an indicator via output device 670 indicating whether autoregulation of patient 410's brain is functioning properly or improperly. For example, monitoring device 430 may output text and or a value on an LCD indicating whether autoregulation of patient 410's brain is working and/or a degree to which the autoregulation mechanism is intact.

In this manner, slow waves induced by ventilator 420 may be used to quickly ascertain whether the state of autoregulation of patient 410's brain. For example, in some instances, medical personnel may be able to determine the state of autoregulation in five minutes or less from the time that the slow waves are introduced to patient 410 (e.g., from the beginning of PEEP oscillation).

Experimental Study

The following experimental study was performed to illustrate concepts consistent with the systems and methodology described above. The study is merely one example consistent with implementations described herein.

A. Ventilation

Neonatal swine (10 in number) were ventilated with a fixed tidal volume of 50 cc at a rate between 15 and 25 cc/kg. Volume control ventilation prevented changes in minute ventilation with varying PEEP. A secondary wave component was introduced into the PEEP control by oscillating PEEP between 5 and 10 cm H₂O in a sine wave pattern with a period of 60 seconds.

B. Signal Sampling and Pressure Reactivity Monitoring

ABP and ICP measurements were recorded every 10 seconds to effectively low-pass filter the ABP and ICP measurements. Pressure reactivity index (PRx) and induced pressure reactivity index (iPRx) (i.e., PRx with PEEP oscillation) values were calculated as a Pearson's coefficient of 30 consecutive samples, defining an analysis epoch at 300 seconds. In addition, the PRx and iPRx values were calculated from overlapping 300 second epochs (i.e., five PEEP wave periods) updated at 60 second intervals to limit the contribution of wave activity slower than 0.003 Hz. In this scenario, the difference between the PRx and iPRx values was considered to be caused by the oscillating PEEP and indicates the presence of hemodynamic activity.

C. Phase Angle Difference Between ABP and ICP

In this experiment, PEEP oscillation occurred at a frequency of 0.0167 Hz (i.e., 60 second period). ΔφAI defines the phase angle difference between ABP and ICP at the frequency of their maximum cross-spectral amplitude between 0.015 and 0.018 Hz to allow for small drift in the PEEP oscillation. The average phase angle difference was calculated from 300 second epochs (five PEEP wave periods) without overlap in the averaging and updated at 60 second intervals. The absolute value of ΔφAI was recorded to prevent phase wrapping at 180°. Each determinant of ΔφAI has a corresponding synchronous value of iPRx. ΔφAI has no meaning without the PEEP oscillation, so it cannot be compared to synchronous traditional PRx measurements. The effects of PEEP oscillation on slow wave activity in the ABP, ICP and central venous pressure (CVP) tracings were quantified by determining the fundamental amplitude of these tracings across the frequency range 0.015 to 0.018 Hz.

D. Analysis

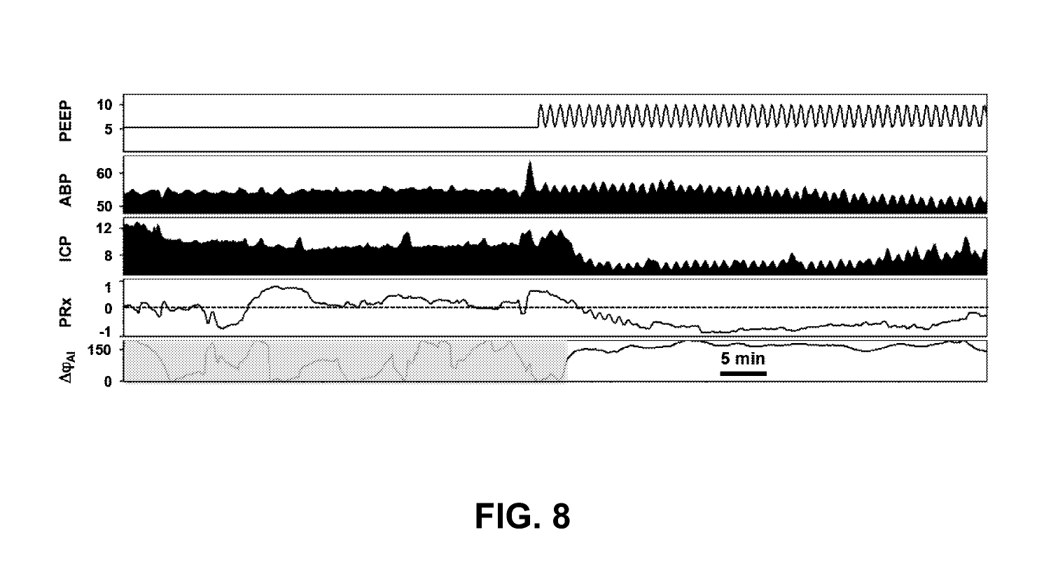

After recovery, at normotension, and without PEEP oscillation, recordings of PRx were made for 60 minutes. This was followed by 60 minutes of iPRx and ΔφAI recordings with PEEP oscillation as described above. FIG. 8 illustrates results associated with comparing PRx, iPRx and ΔφAI in a normotensive, normally autoregulating animal. In FIG. 8, PEEP is shown in cm H₂O; ABP is shown in mm mercury (Hg); ICP is shown in mm Hg; PRx is shown in arbitrary units; and ΔφAI is the phase angle difference between ABP and ICP at PEEP oscillation frequency in degrees (°).

As described above and illustrated in FIG. 8, PEEP oscillated between 5 and 10 cm H₂O after a period of standard ventilation and PEEP of 5 cm H₂O. In this subject, slow wave activity in both the ABP and ICP is erratic until PEEP oscillation begins, at which time both recordings have low amplitude waveforms with the input period of 60 seconds. PRx is unstable and requires a prolonged average to yield a value near zero (0.12 in this recording). iPRx, corresponding to the PRx values once PEEP oscillation begins, is more stable that PRx, (averaging −0.57 in this recording). ΔφAI is not meaningful until the PEEP oscillation has been on for five cycles, and thereafter is a stable value near 150° indicating intact pressure reactivity.

Normotensive newborn piglets normally have robust pressure reactivity and intact cerebrovascular autoregulation. Therefore, the experiment compared the precision of the three metrics in the normal state of pressure reactivity. Precision was quantified for each of the three metrics, in each subject as [median absolute deviation]/[range of possible values] (MAD/RPV). The range of possible values used for the PRx and iPRx was set to range from −1 to 1. The range of possible values for ΔφAI was set to range from 0° to 180° due to the absolute value function applied to prevent phase wrapping at 180°.

E. Accuracy Analysis

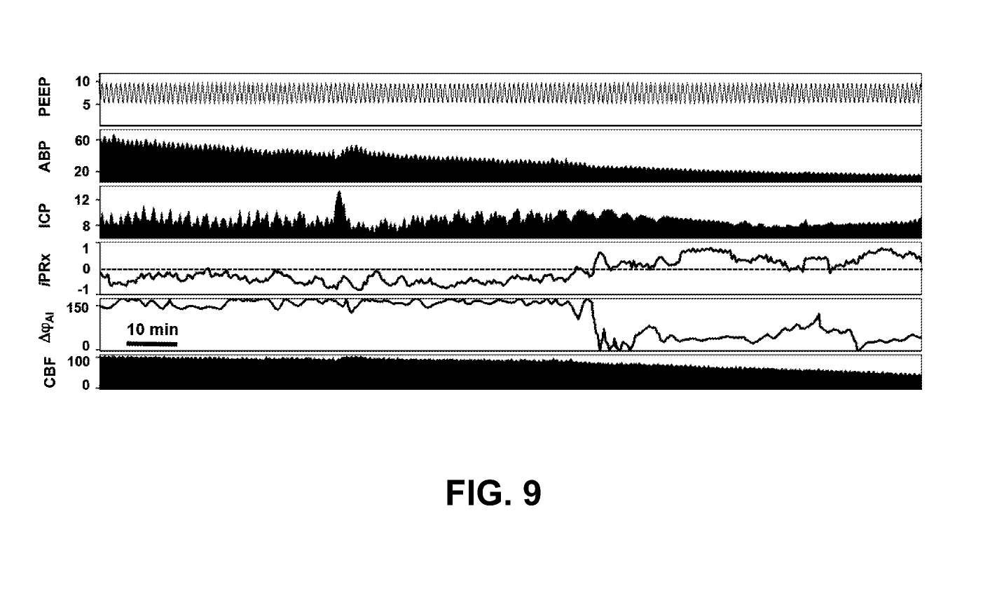

iPRx and ΔφAI were measured in all the animals by continuing the recording through hypotension. PEEP oscillation was left on while the subjects were hemorrhaged by syringe pump withdrawal at a rate of 12% calculated blood volume/hour. This rate provided a graded reduction in ABP to demise over 3-4 hours, as illustrated in FIG. 9. Referring to FIG. 9, iPRx, and ΔφAI were recorded as the lower limit of autoregulation is crossed for a single subject. In FIG. 9, PEEP is measured in cm H₂O; ABP and IPC are measured in mm Hg; iPRx (with oscillating PEEP) is measured in arbitrary units; ΔφAI is measured at PEEP oscillation frequency in degrees (°); and Cerebral Blood flow (CBF) is measured as % Baseline. In this subject, induced slow waves at the PEEP oscillation frequency are seen in the ABP tracing during gradual hemorrhage. Native slow wave activity is evident in the ICP and is slower than the 1/minute PEEP oscillation frequency. A stable negative iPRx (i.e., PRx after PEEP oscillation begins) and a ΔφAI of 150° is seen as ABP is lowered until a critical threshold is crossed, at which time iPRx becomes positive and ΔφAI drops to about 50°.

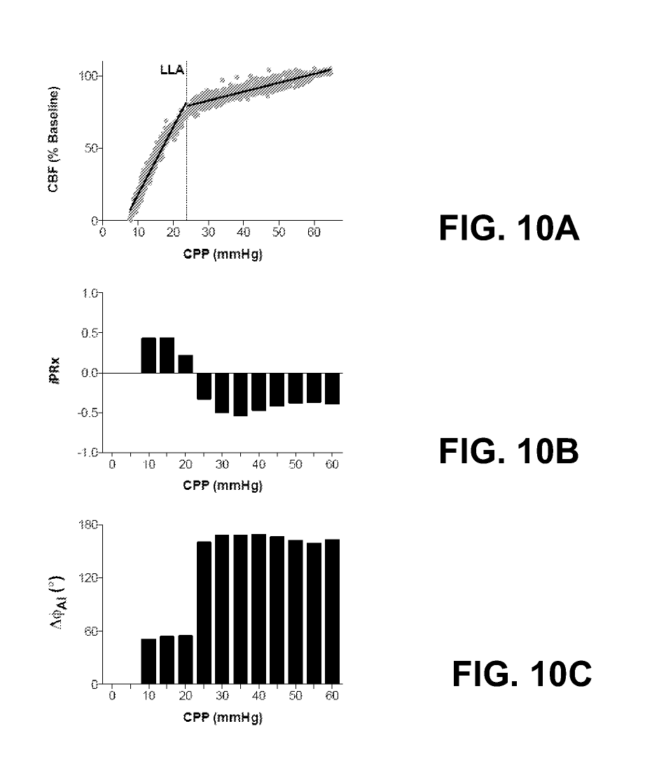

Cortical laser-Doppler flux recordings during hemorrhage were used to delineate the lower limit of autoregulation (LLA). Flux measurements were then plotted across cerebral perfusion pressure and serially dichotomized until rendering the two best-fit lines with lowest combined residual error squared. The intersection of the two lines defines the LLA. This analysis identifies for each subject a single cerebral perfusion pressure above which static autoregulation is intact and below which static autoregulation is impaired. Therefore, the sensitivity and specificity of the dynamic indices iPRx and ΔφAI can be derived by separating data above and below this standard CPP demarcation, as illustrated in FIGS. 10A-10C.

Referring to FIGS. 10A-10C, iPRx and ΔφAI are compared against a standard lower limit of autoregulation (LLA). FIG. 10A illustrates cerebral blood flow (CBF) as a % baseline versus cerebral perfusion pressure (CPP) in mm Hg. FIG. 10B illustrates iPRx in correlation units versus CPP in mm Hg. FIG. 10C illustrates ΔφAI in degrees (°) versus CPP in mm Hg. As illustrated, Laser-Doppler flux recordings are plotted across CPP after normalization to baseline and zero flow. The intersection of two best-fit lines defines the LLA (24 mm Hg in this subject as illustrated in FIG. 10A). iPRx recordings are binned in 5 mm Hg increments of CPP, as illustrated in FIG. 10B, for comparison against the LLA. Negative values above the 25 mm Hg bin indicate intact vascular reactivity. Positive values below the 25 mm Hg bin indicate impaired vascular reactivity. ΔφAI recordings are similarly binned and averaged in FIG. 10C. Above the LLA, there is a stable phase shift of 150°, below the LLA, ΔφAI drops to 50°.

The LLA standard was further validated by verifying a normal static rate of autoregulation (SRoR) across the CPP range of LLA to LLA+15 mm Hg. Laser-Doppler plots were normalized to a percentage of baseline (average flux at a mean CPP 50-60 mm Hg) and biologic zero flux (average flux at demise). Central venous pressure (CVP) was calculated as CPP divided by cortical blood flow (% baseline flux). The slope of CVP plotted across CPP normalized to baseline is the SRoR (% ACVR/% ΔCPP). Values of the static rate of autoregulation when autoregulation is intact are close to 1, and values less than 0.5 indicate impaired autoregulation.

F. Statistics

PRx, iPRx, and ΔφAI were measured serially or synchronously in the same subjects. Therefore, precision was compared for the three metrics accounting for both subject and metric differences with the Freidman test.

To delineate the accuracy of iPRx and ΔφAI, both metrics were categorized and averaged in 5 mm Hg bins of CPP for each subject. CPP was defined as health or disease based on the Doppler-derived determination of LLA. A receiver-operator characteristic test was performed, rendering an area-under ROC curve for each metric.

Variables requiring PEEP oscillation (iPRx, ΔφAI, and the fundamental amplitudes of slow wave activity in the ABP, ICP and CVP recordings) are potentially confounded by changes in cardiac preload. Therefore, all of the PEEP oscillation-dependent variables were examined across three states of preload: normotension, hypotension above the LLA, and hypotension below the LLA using the Freidman test.

Physiologic measurements, blood chemistries, and the ventilating pressures (mean airway pressure (P_{aw} mean) and PIP) were averaged across the following phases of the protocol: normal ventilation, PEEP oscillation, and hemorrhage. These repetitive measures were compared with the Wilcoxon matched-pairs signed rank or Freidman tests where appropriate.

G. Results—Comparing PRx, iPRx, and ΔφAI at Normal ABP

ABP and ICP recordings before PEEP oscillation revealed sporadic slow wave activity. The resultant PRx was −0.06 (−0.16 to 0.03) and demonstrated variability typical of PRx monitoring (median and interquartile range (IQR)). PEEP oscillation caused stable low amplitude variation in both ABP and ICP waveforms. During PEEP modulation, iPRx became constrained around a significantly more negative value of −0.42 (−0.67 to −0.29), more consistent with intact cerebrovascular reactivity (median, IQR, p=0.03 by Wilcoxon matched-pairs signed rank test). ΔφAI was 150° (142° to 160°) during normotension, consistent with intact autoregulation (as described above with respect to FIG. 8).

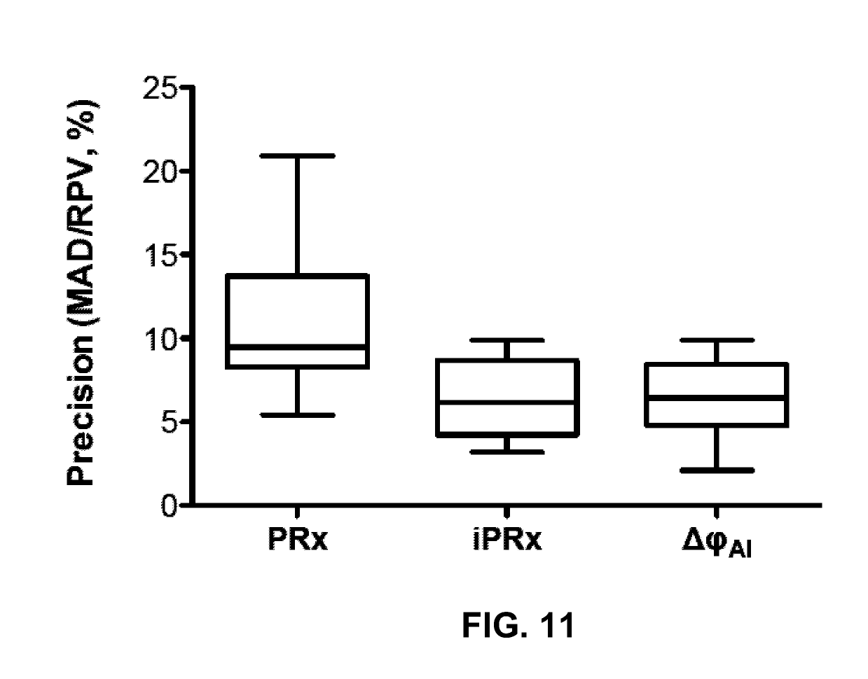

PEEP modulation significantly improved precision of PRx monitoring. MAD/RPV for the PRx, iPRx, and ΔφAI were 9.5% (8.3 to 13.7%), 6.2% (4.2 to 8.7%) and 6.4% (4.8 to 8.4%) respectively (median and IQR; p=0.006 by Friedman's test), as illustrated in FIG. 11. In FIG. 11, the comparison of the precision of PRx, iPRx, and ΔφAI is shown. Referring to FIG. 11, MAD/RPV corresponds to the median absolute deviation (MAD) normalized to the range of possible values (RPV) (%). MAD/RPV was reduced in the iPRx (6.2%; 4.2% to 8.7%) and ΔφAI (6.4%; 4.8 to 8.4%) when compared with traditional PRx (9.5%; 8.3 to 13.7%). Box whiskers are median, IQR and range; P=0.006.

H. Comparing iPRx and ΔφAI Against the Lower Limit of Autoregulation

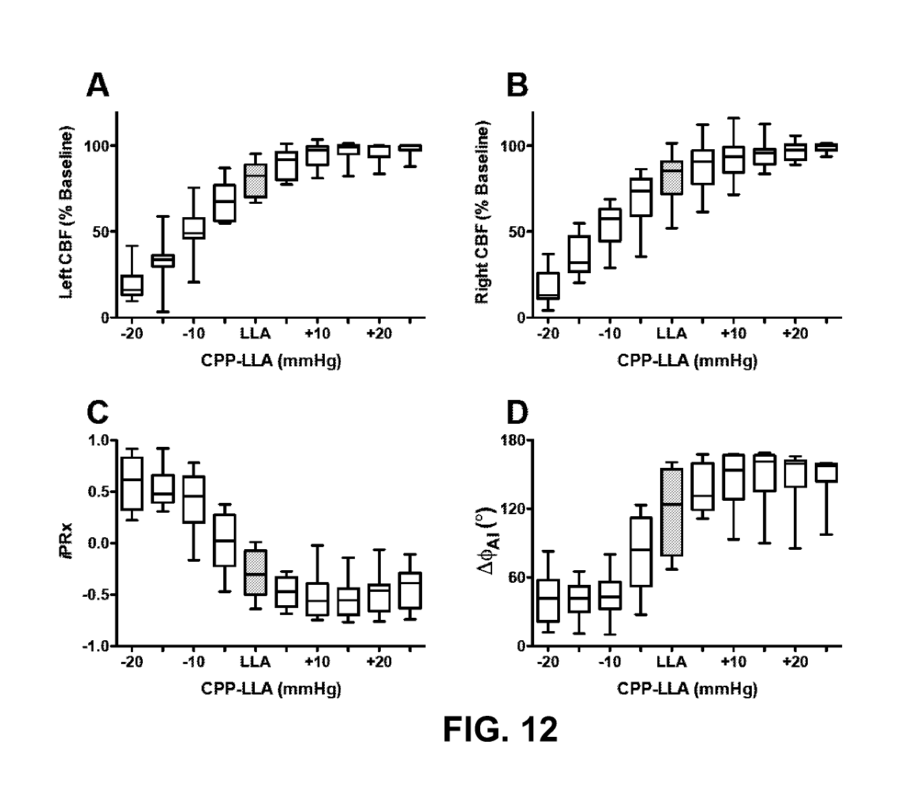

Previous studies comparing PRx against LLA have demonstrated accuracy, and PRx is linked to outcome in multiple studies. This study was not designed to detect a difference in accuracy between PRx, iPRx, and ΔφAI, rather to report the accuracy obtained with PEEP oscillation. The median LLA for the group was 29.7 mm Hg (26.1 to 36.4 mmHg; IQR) and hemispheric differences were small (3.9 mm Hg, 1.2 to 5.9 mm Hg; median, IQR). These values were consistent with previously identified LLA determinations in neonatal swine. Intact autoregulation above LLA was verified by SRoR of 0.79 (0.51 to 0.87; IQR), suitable for defining health in a receiver operator characteristic analysis. CBF, iPRx and PRx are shown normalized to LLA in FIG. 12.

Referring to FIG. 12, normalizing iPRx and ΔφAI to the lower limit of autoregulation is shown in graphs C and D. In FIG. 12, CPP is shown in mm Hg; LLA is shown in mm Hg; CBF is shown as % baseline; iPRx is shown in correlation units; ΔφAI is shown in degrees (°). As illustrated in graphs A and B, cerebral blood flow normalized to LLA gives a visual assessment of the validity of the two best-fit lines method to determine LLA. As further shown in graph C, iPRx values above LLA are negative and iPRx values below the LLA are positive, indicating impaired vascular reactivity. Graph D illustrates that ΔφAI values above the LLA show a large phase angle difference, indicating intact vascular reactivity. Below the LLA, the phase angle is small, indicating pressure passivity.

I. Receiver-Operator Characteristics

Thresholds at 95% sensitivity and 95% specificity for iPRx and ΔφAI were determined. For iPRx, a threshold value of −0.04 was both 95% sensitive and 95% specific for CPP below the LLA. For ΔφAI, a phase angle difference less than 115° was 95% sensitive for CPP below the LLA, and a phase angle difference less than 103° was 95% specific for CPP below the LLA. Areas under receiver operator characteristic curves were 0.988 for both iPRx and ΔφAI.

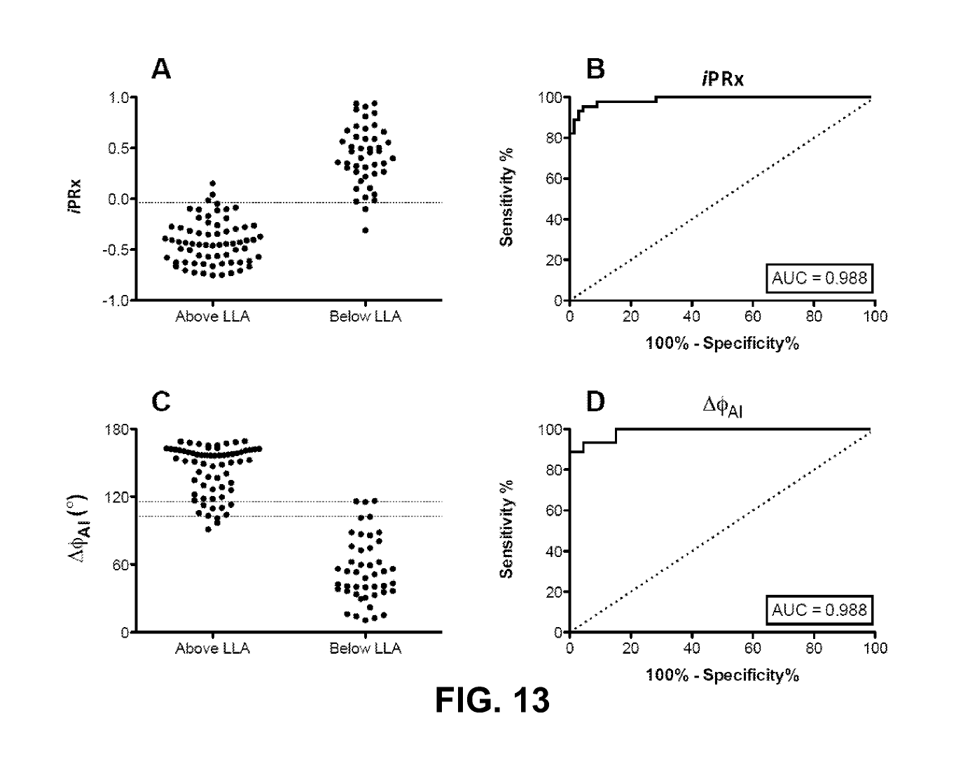

FIG. 13 illustrates the accuracy of iPRx and ΔφAI. In FIG. 13, iPRx is shown in correlation units and ΔφAI is shown in degrees (°). As illustrated in graph A, iPRx of 0.04 (horizontal dashed line) was 95% specific and 95% sensitive for delineating cerebral perfusion pressure (CPP) below the LLA. In graph B, the area under receiver operator characteristic curve (AUC) was 0.988 for the iPRx. In graph C, ΔφAI of 115° was 95% sensitive for delineating CPP below LLA. ΔφAI of 103° was 95% specific for delineating CPP below LLA. In graph D, ΔφAI monitoring yielded the same AUC of 0.988 as iPRx.

J. PEEP-Dependent Variables and Cardiac Preload

The transfer of PEEP amplitude to the fundamental amplitudes of the ABP (a_{ABP}), ICP (a_{ICP}) and CVP (a_{CVP}) was minimally (but statistically significantly) influenced by the state of cardiac preload as shown in Table 3 below.

TABLE 3

| | Normotension | | Hypotension > LLA | | Hypotension < LLA | | P Value |
|---|---|---|---|---|---|---|---|
| a_{ABP} | 3.2 | (2.3 to 4.2) | 4.0 | (3.8 to 5.2) | 3.1 | (2.0 to 4.1) | 0.01 |
| a_{ICP} | 0.43 | (0.25 to 0.48) | 0.51 | (0.31 to 0.60) | 0.24 | (0.17 to 0.29) | 0.02 |
| a_{CVP} | 0.69 | (0.56 to 0.80) | 0.74 | (0.61 to 0.84) | 0.75 | (0.67 to 0.82) | 0.01 |
| iPRx | −0.39 | (−0.49 to −0.33) | −0.42 | (−0.67 to −0.29) | 0.32 | (0.22 to 0.43) | 0.0004 |
| ΔφAI | 150 | (142 to 160) | 161 | (150 to 166) | −31 | (−43 to 12) | <0.0001 |

However, the change in fundamental amplitude of these coherent, induced waves did not affect the phase relationship between ABP and ICP, which is the determinant of both iPRx and ΔφAI. Therefore, iPRx and ΔφAI were not different when comparing the normal preload state and mild hypotension, but hypotension below LLA caused a significantly more positive iPRx, explained by the significantly lower ΔφAI in Table 3. ΔφAI is artificially elevated by the absolute value function needed to control phase wrapping at the limit of 180°. This causes a false increase in ΔφAI when autoregulation is impaired and the value is near zero, but did not impair the ability of ΔφAI to discriminate intact from impaired vascular reactivity. To report the actual phase angle difference between ABP and ICP during impaired autoregulation, a separate, more accurate but impractical calculation of phase angle using a 360° phase limited analysis was done (Table 3).

K. Physiologic Changes with PEEP Oscillation and Hemorrhage

Safe translation of this methodology to clinical practice depends on the clinical impact of PEEP oscillation. The effects of PEEP oscillation and PEEP oscillation during hemorrhagic shock can be seen in the physiologic parameters listed in Table 4 below.

TABLE 4

| | Baseline | | PEEP Oscillation | | Hemorrhage | | P value |
|---|---|---|---|---|---|---|---|
| ABP | 76 | (70 to 83) | 72 | (60 to 78) | n/a | | 0.005 |
| ICP | 9.4 | (7.8 to 12.5) | 10.9 | (7.1 to 12.4) | 10.3 | (6.8 to 13.1) | 0.6 |
| CVP | 4.3 | (2.3 to 5.2) | 4.1 | (3.4 to 7.1) | 4.0 | (3.1 to 5.5) | 0.08 |
| P_{aw} mean | 9.8 | (8.4 to 10.8) | 10.8 | (9.4 to 12.3) | 10.6 | (9.2 to 11.7) | 0.0002 |
| PIP | 17.1 | (14.3 to 19.6) | 18.3 | (15.1 to 20.3) | 16.8 | (13.9 to 18.4) | 0.03 |
| PIP_{PEEP5} | | | 14.4 | (12.2 to 16.4) | | | |
| PIP_{PEEP10} | | | 19.6 | (16.1 to 20.9) | | | |
| pH | 7.43 | (7.34 to 7.45) | 7.46 | (7.43 to 7.49) | 7.47 | (7.42 to 7.48) | 0.07 |
| PₐCO₂ | 39 | (36 to 53) | 39 | (36 to 43) | 38 | (33 to 41) | 0.7 |
| PₐO₂ | 220 | (200 to 246) | 229 | (215 to 263) | 241 | (216 to 256) | 0.4 |
| Hb | 9.8 | (7.5 to 10.5) | 9.7 | (8.4 to 11.1) | 7.5 | (6.7 to 8.4) | 0.0008 |
| Na | 141 | (139 to 142) | 140 | (138 to 143) | 139 | (136 to 143) | 0.2 |

Mean ABP was 76 mmHg (70 to 83 mmHg) before PEEP oscillation and 72 mm Hg (60 to 78 mm Hg) during PEEP oscillation (median, IQR; p=0.05). Although the example displayed in FIG. 8 shows a drop in ICP with initiation of PEEP oscillation, there was no reproducible change in mean ICP with PEEP oscillation. Central venous changes after addition of PEEP oscillation were not significant.

Ventilating pressures changed significantly with PEEP oscillation. All subjects had normal lung compliance. P_{aw} mean increased from 9.8 cm H₂O (8.4 to 10.8 cm H₂O) to 10.8 cm H₂O (9.4 to 12.3 cm H₂O) with addition of PEEP oscillation (median, IQR; p=0.0002). PIP increased from 17.1 cm H₂O (14.3 to 19.6 cm H₂O) at baseline to 18.3 cm H₂O (15.1 to 20.3 cm H₂O) during PEEP oscillation. During oscillation of PEEP, PIP was 14.4 cm H₂O (12.2 to 16.4 cm H₂O) at PEEP 5, and increased to 19.6 cm H₂O (16.1 to 20.9 cm H₂O) at PEEP 10 cm H₂O with a range of 14.4 to 23.9 cm H₂O (median, IQR; p<0.0001).

None of the arterial blood gas trends across phases of the experiment were significant. Arterial hemoglobin concentration dropped during hemorrhage: 9.8 mg/dL at baseline (7.5 to 10.5), 9.7 mg/dL during PEEP oscillation (8.4 to 11.1), and 7.5 mg/dL (6.7 to 8.4 mg/dL) during hemorrhage (median, IQR, p=0.0008).

Cerebral vascular reactivity monitoring performed in the manner discussed above allows medical personnel to be informed of a fundamental variable of care for patients with brain injury: where to target cerebral perfusion pressure (CPP). In this particular methodology, monitoring cerebrovascular autoregulation is performed by inducing low amplitude ABP waves with a slow PEEP modulation. In addition, the methodology described herein effectively separates the respiratory function of the ventilator from the autoregulation interrogation function by, for example, providing programming via a control device to provide a slow wave component via the ventilator. This slow wave component does not interfere with the ventilator's normal functions (e.g., oxygenating and ventilation/CO₂ removal), is adjusted to be slower than respiration and is within the bandwidth of Lundberg's B waves. Consistent, low amplitude ABP and ICP waves resulted, persistent across a range of cardiac preload states. The phasic relationship between these coherent ABP and ICP waves was predictive of the state of autoregulation. Intact and impaired autoregulation were distinguished by a separation of, for example, a 192° phase angle difference between ABP and ICP (128° to 204°, median IQR).

In summary, in implementations described above, mean airway pressure oscillations may be created at a low frequency to produce corresponding oscillations in arterial blood pressure. Phase angle analysis of the oscillations with respect to arterial blood pressure and cerebral blood volume may then be analyzed. It has been found that if a phase angle difference is present, autoregulation is intact or partially intact. The phase angle analysis has proven to be robust in its ability to delineate pressure-reactive from pressure-passive states in the cerebral vasculature.

### CONCLUSION

Implementations described herein provide repetitive, hemodynamic oscillations by inducing variations of the mean airway pressure via a ventilator. These induced slow waves allow for precise measurements with respect to autoregulation in a very short period of time. The slow waves may also be induced without interfering with the ventilation functions of the ventilator. In addition, cerebral vascular reactivity monitoring performed in the manner described herein may allow medical personal to quickly ascertain where to target CPP for the patient, which a fundamental variable of care for patients with brain injury.

The foregoing description of exemplary implementations provides illustration and description, but is not intended to be exhaustive or to limit the invention to the precise form disclosed. Modifications and variations are possible in light of the above teachings or may be acquired from practice of the invention.

For example, various features have been described above with respect to various devices performing various functions. In other implementations, the functions described as being performed by a particular device may be performed by another device. In addition, functions described as being performed by a single device may be performed by multiple devices, or vice versa.

Still further, an experimental study involving swine has been described. This study is merely provided as an illustrative example of the viability of aspects of the invention described herein.

It will be apparent to one of ordinary skill in the art that various features described above may be implemented in many different forms of software, firmware, and hardware in the implementations illustrated in the figures. The actual software code or specialized control hardware used to implement the various features is not limiting of the invention. Thus, the operation and behavior of the features of the invention were described without reference to the specific software code—it being understood that one of ordinary skill in the art would be able to design software and control hardware to implement the various features based on the description herein.

Further, certain portions of the invention may be implemented as “logic” that performs one or more functions. This logic may include hardware, such as a processor, a microprocessor, an application specific integrated circuit, or a field programmable gate array, software, or a combination of hardware and software.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

What is claimed is:

1. A method, comprising:
controlling a ventilator to introduce mean airway pressure (MAP) variations in a patient to induce slow waves of substantially fixed amplitude and period to the patient;
analyzing arterial blood pressure in the patient with respect to the MAP variations; and
determining, based on the analyzing, whether an autoregulatory mechanism associated with the patient's brain is operating properly.

2. The method of claim 1, wherein the analyzing is performed at a frequency corresponding to a frequency of the slow waves.

3. The method of claim 2, wherein a frequency associated with the MAP variations is less than 0.1 hertz (Hz) and the analyzing is performed at the frequency of the MAP variations.

4. The method of claim 2, wherein the determining comprises:
determining whether peak blood volume in the brain is phase shifted with respect to the arterial blood pressure.

5. The method of claim 1, wherein the determining comprises:
comparing arterial blood pressure of the patient to intracranial pressure of the patient in a frequency domain, and
identifying a phase angle difference between the arterial blood pressure and the intracranial pressure.

6. The method of claim 5, wherein the determining further comprises:
determining that the autoregulatory mechanism is functioning properly in response to identifying that the phase angle difference is within a predetermined range.

7. The method of claim 1, wherein the controlling a ventilator comprises:
controlling positive end-expiratory pressure (PEEP) provided to the patient to vary PEEP over a period of time.

8. The method of claim 7, wherein the controlling a ventilator further comprises:
controlling the ventilator to provide a fixed tidal volume to the patient while simultaneously varying PEEP.

9. The method of claim 8, wherein the determining further comprises:
determining that the autoregulatory mechanism is operating properly in response to determining that the peak blood volume in the brain is negative phase shifted in a frequency domain with respect to the arterial blood pressure.

10. A system, comprising:
a ventilator configured to:
provide ventilation functions to a subject, and
provide mean airway pressure (MAP) variations to the subject to induce slow waves to the subject, wherein the slow waves have a fixed amplitude and frequency and are provided simultaneously with the ventilation functions.

11. The system of claim 10, further comprising:
at least one monitoring device configured to:
analyze arterial blood pressure of the patient with respect to the MAP variations, and
determine, based on the analyzing, whether an autoregulatory mechanism associated with the subject's brain is operating properly.

12. The system of claim 11, wherein when analyzing arterial blood pressure, the at least one monitoring device is configured to:
analyze the arterial blood pressure at a frequency corresponding to the frequency of the slow waves.

13. The system of claim 12, wherein a frequency associated with the MAP variations is less than 0.1 hertz (Hz) and the at least one monitoring device is configured to analyze the arterial blood pressure at the frequency of the MAP variations.

14. The system of claim 11, wherein when determining, the at least one monitoring device is configured to:
determine whether peak blood volume in the brain is phase shifted with respect to the arterial blood pressure.

15. The system of claim 11, wherein when determining, the at least one monitoring device is configured to:
compare arterial blood pressure of the patient to intracranial pressure of the subject in a frequency domain,
identify a phase angle difference between the arterial blood pressure and the intracranial pressure, and
output information indicating that the autoregulatory mechanism is functioning properly in response to identifying that the phase angle difference is within a predetermined range.

16. The system of claim 10, wherein the ventilator includes a positive end-expiratory pressure (PEEP) controller, and when providing MAP variations to the subject, the PEEP controller is set to oscillate PEEP between a first value and a second value over a period of time and to repeat the varying for a predetermined duration.

17. The system of claim 16, wherein the ventilator includes a volume controller, and wherein the volume controller is set to provide a fixed tidal volume to the subject while simultaneously oscillating PEEP.

18. A computer-readable medium having stored thereon sequences of instructions which, when executed by at least one processor, cause the at least one processor to:
control a ventilator to introduce mean airway pressure (MAP) variations to generate slow waves to a patient, wherein the slow waves have a fixed amplitude and frequency;
analyze arterial blood pressure in the patient at a frequency corresponding to the slow wave frequency; and
determine, based on the analyzing, whether an autoregulatory mechanism associated with the patient's brain is operating properly.

19. The computer-readable medium of claim 18, wherein when analyzing, the instructions cause the at least one processor to:
compare arterial blood pressure of the patient to intracranial pressure of the patient at the slow wave frequency, and
identify a phase angle difference between the arterial blood pressure and the intracranial pressure.

20. The computer-readable medium of claim 18, wherein controlling a ventilator to introduce MAP variations, the instructions cause the at least one processor to:
signal the ventilator to oscillate PEEP between a first value and a second value over a period of time and to repeat the varying for a predetermined duration.
